(19) United States
(12) Patent Application Publication
(10) Pub. No.: US 2012/0095058 A1
(43) Pub. Date: Apr. 19, 2012

(54) Method of preventing survival of retrovirally cells and of inhibiting formation of infectious retroviruses

(76) Inventors: H., M. Hanauske-Abel, Englewood Cliffs, NJ (US); Paul Palumbo, Westfield, NJ (US); B., M. Cracchiolo, Englewood Cliffs, NJ (US); Myung-Hee Park, Potomac, MD (US); Edith Wolff, Bethesda, MD (US); Axel-Rainer Hanauske, Frankfurt (DE); Michael B. Mathews, Montclair, NJ (US); Deepti Saxena, South Orange, NJ (US); Mainul Hoque, Newark, NJ (US)

(21) Appl. No.: 10/581,658

(22) PCT Filed: Dec. 1, 2004
(86) PCT No.: PCT/US2004/040236

§ 371 Date: Sep. 2, 2011

Related U.S. Application Data
(60) Provisional application No. 60/526,110, filed on Dec. 1, 2003.

(51) Int. Cl. A61K 31/4418; A61K 31/4439; A61K 31/4412; A61P 31/18; A61P 31/14; C12N 5/071; A61K 31/426; A61P 31/12

(52) U.S. Cl. 514/342; 514/345; 514/365; 435/375; 514/348

(57) ABSTRACT

The present invention discloses compounds and pharmaceutical compositions which are highly effective at inhibiting the accumulation of spliced and unspliced viral transcripts and their utilization for viral protein synthesis at cellular ribosomes, and at inhibiting the formation of the hypusine residue in cellular eIF-5A precursor proteins, the cellular cofactors that render spliced and unspliced viral transcripts translatable at the ribosomes of infectled cells. The invention further relates to methods of using such compounds and pharmaceutical compositions therefrom for inhibiting or preventing viral protein synthesis. Such inhibition cause a dose-dependent release from the virally induced arrest of the otherwise genetically preprogrammed apoptosis of virally infected cells, and in consequence, triggers their apoptotic ablation and the eradication of the chronic infection-mediating provirus integrated into their genome.

### BACKGROUND

Viruses require cells for their multiplication. In an infected cell, they achieve this goal by parasitizing and hijacking metabolic pathways, especially those involved in the synthesis of proteins and nucleic acids. Over evolutionary times, cells have developed mechanisms that detect the hijacking of these synthetic pathways by viral intruders. When triggered, these mechanisms activate the invaded cell to initiate and complete self-destruction. The suicide of individual cells is intended to curb the spread of the viral intruder by destroying the production sites that release the invader in large numbers. All viruses therefore have developed molecules that inhibit the suicide of infected cells, in this way securing their continued enslavement as viral generators. Such antiapoptotic viral molecules are formed inside invaded cells as part of the viral takeover of the cellular synthetic machinery, which at the same time is also generating the infective viruses themselves.

Lentiviruses use a particular set of viral proteins, the Rev/Rex class, to transport certain viral mRNA, generally those that are incompletely spliced and thus would be retained in the nucleus of an infected cell, from the nuclei of infected cells to the polysomes of infected cells. These Rev/Rex-dependent mRNAs display specific nucleotide motifs for binding to Rev/Rex. Rev/Rex-dependent viral mRNAs are generally categorized as occurring late in the infection cycle and contain intronic sequences that otherwise would render them ineligible for translation into viral protein, e.g by denial of nuclear export, yet they encode proteins essential for suppression of apoptosis, virion formation and infectivity.

One of the pivotal cellular partners for the Rev/Rex proteins is eukaryotic translation initiation factor 5A (eIF5A), of which two isoforms with minor differences in sequence are known at present. Both contain the unique, genetically not encoded residue hypusine [N⁶-(4-amino-2(R)-hydroxybutyl)-L-lysine]. Hypusine is essential for the biological function of the eIF5A proteins, which includes the nucleocytoplasmic transport and translatability of proliferation-associated cellular mRNAs. Hypusine is formed through two consecutive posttranslational enzymatic modifications of a specific, genetically encoded lysine side chain of the genetically encoded eIF-5A precursor protein. In the first step, catalyzed by deoxyhypusine synthase (DOHS), the intermediate deoxyhypusine form of eIF5A is generated the nicotinamide adenine dinucleotide (NAD)-dependent transfer of the 4-aminobutyl moiety of the polyamine spermidine to the terminal amino group of a specific lysine residue in the eIF-5A precursor. The product-forming modification is a hydroxylation, mediated by deoxyhypusine hydroxylase (DOHH), a 2-oxoacid utilizing dioxygenase like all other known protein hydroxylases. In human cells infected with the human immunodeficiency virus type 1 (HIV-1), the 3-hydroxypyrid-4-one class and the isomeric 3-hydroxypyrid-2-one class of DOHH inhibitors, in the range of 100-200 μM, decease the availability of mature, hypusine-containing eIF5A and thus of the functional partner for the Rev protein of HIV-1. This, in turn, results in suppression of infective virion formation, disruptiion of the synthesis of the major capsid proteins, and release from the retrovirally mediated apoptosis arrest (Hanauske-Abel et al., U.S. Pat. No. 5,849,587, which is hereby incorporated by reference; Andrus et al. Biochem. Pharmacol. 55, 1807-1818, 1998, which is hereby incorporated by reference).

### SUMMARY

The present invention discloses compounds and pharmaceutical compositions which are highly effective at inhibiting the accumulation of spliced and unspliced viral transcripts and their utilization for viral protein synthesis at cellular ribosomes, and at inhibiting the formation of the hypusine residue in cellular eIF-5A precursor proteins, the cellular cofactors that render spliced and unspliced viral transcripts translatable at the ribosomes of infected cells. The invention further relates to methods of using such compounds and pharmaceutical compositions therefrom for inhibiting or preventing viral protein synthesis. Such inhibition causes a dose-dependent release from the virally induced arrest of the otherwise genetically preprogrammed apoptosis of virally infected cells, and in consequence, triggers their apoptotic ablation and the eradication of the chronic infection-mediating provirus integrated into their genome.

While not wishing to be bound by theory, it appears the structure and use of compounds of the present disclosed invention prevent the viral take-over of the cellular synthetic machinery and limit the viral spread through, and persistence in, the various susceptible cell populations of an individual. The translational disruption of viral protein formation causes loss of anti-apoptotic effect, resulting in reactivation of the apoptotic self-destruction of infected cells. The compounds of this invention likewise cause a general decrease in the viral proteins required for the assembly of infective viruses. The knowledge of the cellular proteins, in particular of the eIF5A system, that renders incompletely spliced viral mRNAs translatbale at cellular ribosomes, can be used for the identification of novel antiviral compounds which promote the apoptotic clearance preferentially or exclusively of virally infected cells in a living system. The administration of such compounds and composition can be employed clinically to break the cycle of viral infectivity within a living system and among living systems.

The present invention is directed to therapeutic compositions and methods that employ compounds and composition that inhibit the posttranslational formation of hypusine, which thereby inhibits the formation of the bioactive intracellular protein eIF-5A. This inhibition may be used for suppressing infections by retroviruses that rely on parasitizing the eIF5A system so as to secure and promote their own replication and infectious propagation. The methods of the present invention involve administering, to eukaryotic cells, tissues, or individuals, compounds which block the posttranslational intracellular formation of hypusine, in an amount effective to suppress synthesis of bioactive eIF-5A or its bioactive isoforms; suppress translationally productive recruitment of of said eIF-5A or its isoforms by retroviral elements of nucleic acid and/or protein structure; inhibit translation of anti-apoptotic retroviral proteins from retroviral transcripts; and induce apoptotic ablation of retrovirally-infected eukaryotic cells.

One embodiment of the invention relates to a method of inhibiting intracellular translation of viral mRNAs into viral proteins required for virion assembly and infectivity, comprising administering, to eukaryotic cells, tissues, or individuals, an agent which blocks the accumulation ofspliced and unspliced viral transcripts and their utilization for viral protein synthesis at cellular ribosomes.

Another embodiment of the invention relates to a method of inhibiting the utilization of spliced and unspliced viral transcripts for viral protein synthesis at cellular ribosomes comprising administering, to eukaryotic cells, tissues, or individuals, an agent which blocks hypusine formation within eIF5A in an amount sufficient to suppress the translationally productive interaction of eIF-5A with viral elements of nucleic acid and/or protein structure.

Another embodiment of the invention relates to a method of inhibiting synthesis of specific viral proteins of Rev/Rex-dependent lentiviruses, or of viruses dependent on interaction of eIF-5A with viral elements of nucleic acid and/or protein structure comprising administering, to eukaryotic cells, tissues, or individuals, an agent which blocks hypusine formation and thus eIF5A function in an amount sufficient to inhibit biosynthesis of viral proteins of Rev/Rex-dependent lentiviruses or of viruses dependent on interaction of eIF-5A with viral elements of nucleic acid and/or protein structure.

Yet another embodiment of the invention relates to a method of inhibiting replication of Rev/Rex-dependent lentiviruses, or viruses dependent on interaction of eIF-5A with viral elements of nucleic acid and/or protein structure comprising administering, to eukaryotic cells, tissues, or individuals, an agent which blocks hypusine formation and thus eIF5A function or reduces the availability of Rev/Rex protein, in an amount sufficient to inhibit replication of Rev/Rex-dependent lentiviruses or of viruses dependent on interaction of eIF-5A with viral elements of nucleic acid and/or protein structure.

Yet another embodiment of the invention relates to a method of inducing apoptosis in cells infected with Rev/Rex-dependent lentiviruses or viruses dependent on interaction of eIF-5A with viral elements of nucleic acid and/or protein structure comprising administering, to cells infected with such viruses, an agent which blocks intracellular hypusine formation or reduces the availability of Rev/Rex protein, in an amount sufficient to induce apoptotic ablation of virally-infected cells.

Another embodiment relates to a method for suppressing genital transmission of human immunodeficiency virus which comprises administering to a male or female genital a compound of formula III or IV

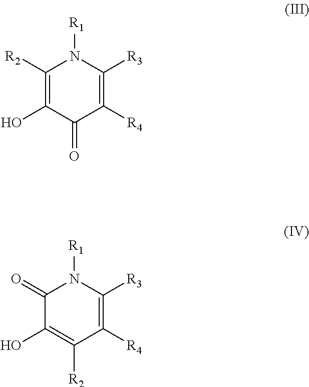

wherein R₁, R₂, R₃, and R₄ each individually represent a hydrogen, an alkyl, alkenyl or alkoxy group containing 1 to about 8 carbons, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

One embodiment of the present invention involves inducing apoptosis in eukaryotic cells infected with Rev/Rex-dependent retroviruses that rely, for multiplication and infective propagation, on the availability in host cells of mature eIF-5A for interaction with viral elements of nucleic acid and/or protein structure. Induction of apoptosis is achieved by administering a compound or a composition thereof to eukaryotic cells which blocks the posttranslational intracellular formation of hypusine, i.e. the maturation of eIF5A, in an amount sufficient to ablate virally-infected cells.

One embodiment of the present invention is a therapeutic composition comprised of a N-hydroxypyrid-2-one compound of formula (I) and derivatives thereof (including salts, tautomeric forms, and solvates) or a pharmaceutical composition including the compounds of

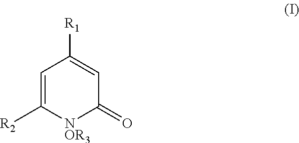

wherein R₁ is (C1-C6) alkyl; R₂ is (C1-C10) straight or branched alkyl, (C3-C6)cycloalkyl or phenoxy(C1-C3)alkyl, where the phenoxy group is substituted by substituted or unsubstituted phenoxy; and R₃ is hydrogen or a pharmacologically acceptable salt. Preferably R₁ is methyl.

More preferably, R₁ is methyl, R₂ is cyclohexyl or

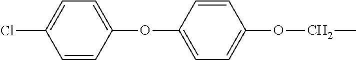

and R₃ is hydrogen.

In another embodiment, R₁ is methyl, R₂ is (CH₃)₃CCH₂(CH₃)CH₂— and R₃ is ⁺H₃NCH₂OH.

Examples of such compound and salts thereof of formula I useful in the practice of the present invention include, but are not limited to, ciclopirox (CAS #29342-05-0), rilopirox (CAS #104153-37-9) and their analogs, such as metipirox (CAS #29342-02-7) or piroctone [CAS #506050-76-5]); as well as their (1:1) ethanolamine salts, exemplified by octopirox (CAS #68890-6-4).

Another embodiment of the present invention is a therapeutic composition comprised of a thiazoline-4-carboxylic acid compound of formula (II) and derivatives thereof (including salts, tautomeric forms, and solvates) or a pharmaceutical composition including the compounds of

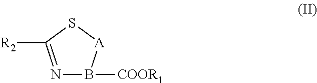

where R₁ is hydrogen or a pharmacologically acceptable salt; R₂ is ortho hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen, or hydroxyl; and A-B is —CH₂—CR₃— or —CH═CH—, where R₃ is hydrogen or (C₁-C₆)alkyl.

In one embodiment of the invention R₁ is hydrogen, R₂ is phenyl, A-B is —CH═CR₃—, and R₃ is hydrogen.

More preferably R₂ is 2-hydroxy-4-methylphenyl or 2,4-dihydroxyphenyl. In another embodiment of the invention, R₁ is hydrogen and R₂ is pyridyl. More preferably, R₂ is 3-hydroxy-2-pyridyl and A-B is —CH₂—C(CH₃)— or —CH═CH—.

In another embodiment of the invention R₁ is hydrogen, R₂ is phenyl, A-B is —CH═CR₃— and R₃ is hydrogen. More preferably R₂ is 2-hydroxy-4-methyl.

The virally or retrovirally infected cells to which the compound or its compositions are administered may be present in vivo or in vitro; in a fluid; in a tissue; in an organ; or in an individual. The administration may include cycled or non-cycled, high- or low-dose, pulse or continuous administration modes of a compound, or alone or in combination with other compunds of this invention or in combination with other antiviral or antiretroviral compounds, exemplified by cycled high-dose pulse administration of a compound of this invention in the presence of continued medication with an antiretroviral drug in current clinical use.

The compound or composition administered to the virally or retrovirally infected cells interferes with the catalytic activity of the enzyme deoxyhypusine hydroxylase, which is indispensible for the formation of the functionally essential hypusine residue of mature eIF-5A. By disrupting the formation of bioactive eIF5A, the viral invadors are denied access to the translational machinery of an infected and cannot reprogram its performance to serve their replicative interest. Since a cellular element rather than a viral one is the target of this therapeutic intervention, and since the cellular element is required for the translational processing of essential yet incompletely spliced viral transcripts, the virus cannot develop escape mutations without reshaping its entire genome and mode of replication.

### DESCRIPTION OF THE DRAWINGS

FIG. 1. Ciclopirox reduces gene expression from an HIV-1 molecular clone.

(A, B) Inhibition of accumulation of spliced and unspliced HIV transcripts. Human 293 T cells were co-transfected with plasmids carrying the HIV-1 molecular clone (10 μg) and a CMV-Renilla luciferase construct (2 μg). Ciclopirox (CPX) or Princeton 2 (P2) were added to a final concentration of 30 μM 3 hours later. RNA was isolated from nuclear and cytoplasmic fractions at 15 hours after transfection. RNase protection assays used probes for the HIV-1 major splice donor site sequence (A), and Renilla luciferase (B). Arrows indicate the positions of protected probe fragments corresponding to unspliced (US) and spliced (S) HIV-1 RNA and Renilla luciferase (R-Luc). Lanes marked ‘Probe’ contain undigested probe RNA equivalent to 10% of the input to protection assays. The radioactivity of the protected bands, quantified with a phosphoimager using ImageQuant, is expressed in relative units.

(C) Specificity of inhibition by CPX. Cells were co-transfected with 500 ng pNL4-3-Luc E-, or with 100 ng of HIV-1 LTR-firefly luciferase reporter plasmid and 20 ng of pRSV-Tat, together with 100 ng of CMV-Renilla luciferase reporter plasmid. Buffer (Control), 30 μM ciclopirox (CPX), or 30 μM Princeton 2 (P2) was added during transfection, and cells were harvested at 12 hours post-transfection for luciferase assay. Results are presented as the ratio of firefly luciferase to Renilla luciferase, normalized to the controls, with standard deviations indicated.

FIG. 2 Summary of the biosynthetic pathway that constitutes the posttranslational modification of the lysine-containing, immature form of eIF5A into the hypusine-containing, mature one.

FIG. 3 Inhibition of purified DOHH, the hypusine-forming enzyme, by ciclopirox (CPX) and Princeton 2 (P2).

FIG. 4 Inhibition of hypusine formation in HIV-1-infected H9 cells by ciclopirox (CPX) and Princeton 2 (P2).

FIG. 5 Inhibition by ciclopirox (CPX) and Princeton 2 (P2). of p24 formation in HIV-1-infected H9 cells.

FIG. 6 Inhibition by ciclopirox (CPX) and Princeton 2 (P2) of p24 formation by freshly isolated peripheral blood mononuclear cells, acutely infected with infected with a patient-isolate of HIV-1.

FIG. 7 Inhibition by ciclopirox (CPX) and Princeton 2 (P2) of HIV-1 RNA formation by freshly isolated peripheral blood mononuclear cells, acutely infected with infected with a patient-isolate of HIV-1.

FIG. 8 Induction of apoptosis by ciclopirox (CPX) and Princeton 2 (P2) of HIV-1 RNA formation by freshly isolated peripheral blood mononuclear cells, acutely infected with infected with a patient-isolate of HIV-1.

FIG. 9 Inhibition by deferiprone of HIV-1 protein (p24) formation, HIV-1 RNA formation, and of HIV-1 DNA by freshly isolated peripheral blood mononuclear cells, chronically infected with a patient-isolate of HIV-1.

### DETAILED DESCRIPTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” or a “virus” is a reference to one or more cells and equivalents thereof, or viruses and the various representations thereof, as known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

The present invention is directed to methods of inhibiting the utilization of spliced, incompletely spliced and unspliced viral transcripts for viral protein synthesis at cellular ribosomes. The translational utilization of said viral transcripts involves the mature, i.e. hypusine-containing form of the cellular eukaryotic translation initiation factor 5A (eIF5A) proteins. Their genetically non-coded residue hypusine [N6-(4-amino-2(R)-hydroxybutyl)-L-lysine] imparts bioactivity and is synthesized from a specific, protein-bound lysine by two sequential posttranslational modification, the final and irreversible, product-forming step being catalyzed by deoxyhypusine hydroxylase (DOHH), as shown in FIG. 2. The present invention involves inhibiting intracellular synthesis of mature and bioactive eIF-5A, without suppressing the synthesis of its genetically encoded, lysine-containing precursor. The mature and bioactive, hypsuine-containing form of eIF-5A is involved in enabling translation of viral transcripts, in particular the incompletely spliced and unspliced species, for viral protein synthesis at cellular ribosomes, presumably by forming complexes with nucleic acids and/or viral proteins. These methods include administering, to eukaryotic cells, tissues, or individuals, a composition which in a sufficient amount inhibits the posttranslational intracellular formation of hypusine inside the lysine-eIF-5A precursor.

Another aspect of the present invention involves inducing apoptosis in eukaryotic cells infected with Rev/Rex-dependent viruses, or viruses dependent on translational activation by mature host-cell eIF-5A of viral nucleic acids which encode proteins that inhibit execution of the genetically preprogrammed suicide (apotosis) of virally infected cells, in effect extending their availability as virion production facilities. Antiapoptotic viral proteins encoded by Rev/Rex-dependent viruses are know in the art, as exemplified by the Vpr protein of HIV-1 (Fukumori et al., FEBS Lett 432, 17-20, 1998, which is hereby incorporated by reference). Inducing apoptosis in infected cells is achieved by administering a compound of formulas (I), (II), or (III) to eukaryotic cells infected with Rev/Rex-dependent viruses that dependent on the interaction of mature, hypusine-containing eIF-5A with viral elements of nucleic acid and/or protein structure.

Retroviruses, of which lentiviruses are a genus, are typified by the human immunodeficiency virus type 1 (HIV-1). They share the strict requirement for a specific regulator system (i.e. the Rev/Rex protein and its nucleic acid response element termed ‘RRE’) in order to express viral structural and functional genes and. hence, to propagate efficiently and produce infectious progeny. In addition to the human immunodeficiency viruses, this group consists of, but is not limited to, human T-cell leukemia viruses, hepatitis B virus, visna virus, simian immunodeficiency viruses, bovine immunodeficiency virus, equine infectious anemia virus, feline immunodeficiency viruses, caprine arthritis-encephalitis virus, and Mason Pfizer virus. Reference to HIV-1 is used here as a non-limiting example to exemplify the function of this regulator system, to delineate its interaction with mature host-cell eIF-5A, and to demonstrate the methods of this invention as they are applied to interfere with this system by denying it a bioactive cellular cofactor and thus, rendering it nonfunctional.

Retroviruses use a particular set of viral proteins, of the Rev/Rex class, to transport incompletely spliced and unspliced viral transcripts from the nucleus of infected cells to the polysomes in the cytoplasm of the cell for translational utilization. These mRNAs display specific nucleotide motifs for binding to Rev/Rex. Rev/Rex-dependent viral mRNAs are generally categorized as occurring late in the infections cycle, contain intronic sequences that otherwise would block their nuclear export and translational utilization, and encode major proteins essential for virion formation, infectious propagation, and arrest of apoptosis in infected cells.

With respect to the present inventive methods, the tissue can be a tissue of any living organism such as a mammal, and may be treated in vivo, in vitro, or ex vivo. The term “in vivo” as used herein means that the tissue and the cells are found within a living system. The term “ex vivo” as used herein means that the tissue and the cells are derived from a living system but taken out of the living system. The term “in vitro” as used herein means that the tissue and the cells are maintained in an appropriate culture system. Mammals may include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

After viral infection of human cells by a retrovirus such as but not limited to HIV-1, the viral genomic RNA is transcribed into DNA and subsequently incorporated into the human genome as provirus, the latter becoming the original template for the on-going production of viral transcripts including viral genomic RNA. Upon transcription, only the completely spliced 2-kb class of transcripts encoding the HIV -1 proteins Tat, Rev, and Nef, or Tat-Rev fusion proteins, are exported into the cytoplasm, where they are efficiently translated by the protein-producing machinery of the host cell. The incompletely spliced 4-kb class and the unspliced 9-kb class of viral transcipts cannot by themselves be exported and, thereby, fail to gain productive access to this machinery, due to control mechanisms that in eukaryotes generally deny translation of incompletely spliced and unspliced RNA. This failure to be exported, apparently due to lack of nucleocytoplasmic transport and/or polysomal access, is of grave consequence to the replicative ability of retroviruses like HIV-1 and would severely limit production of any virions. Not only are all the structural and antiapoptotic proteins of HIV-1 encoded by these incompletey/unspliced transcripts, but the 9-kb class also constitutes the infectious viral genome to be packaged into these particles. It is the function of the Rev/RRE system of HW-1 to overcome this blockade, with Rev re-entering into the nucleus after being synthesized on cytoplasmic host-cell polysomes, binding to the Rev-response element (“RRE”) of the 4-kb and 9-kb transcripts, assembling a further complex with cellular partner proteins including mature eIF5A and, thereby, rendering these transcipts eligible for entry into the cytoplasm and for acess to the protein producing machinery of the host cell, resulting in biosynthesis of the major viral proteins, in particular Gag, Pol, Vif, Vpr, Vpu, and Env.

The mature, hypusine-containing eIF-5A protein is the critical element in a proposed pathway that provides preferential polysomal access to a subclass of specific cellular mRNAs which encode proteins that enable and coordinate DNA replication, i.e., initiate cellular proliferation. This subset has been termed hypusine-dependent messenger nucleic acids, or hymns (Hanauske-Abel et al., FEBS Left. 366, 92-98, 1995, which is hereby incorporated by reference). In this way, eIF-5A enables preferential polysomal loading of the estimated only about 120 different mRNAs of the hymns type and directly entitles them to translation, bypassing the need to “wait in line” until ribosomes become available. The hymnsencoded proteins, in turn, are thought to be essential for irreversibly engaging the multi-component machinery that initiates replication of eukaryotic cells (Hanauske-Abel et al., FEBS Lett. 366, 92-98, 1995, which is hereby incorporated by reference). Translation of the vast majority of cellular mRNAs, estimated to reach over 20,000 distinct species per cells, is bypassed. The routine translation of all these mRNAs constitutes the usual mechanism for “housekeeping” protein biosynthesis and proceeds independent of hypusine formation and the eIF-5A pathway.

The Rev protein of HIV-1 is known to bind to the mature eIF-5A of infected host cells (Ruhl et al., J. Cell. Bio. 123,1309-1320, 1993, which is hereby incorporated by reference). In this way, the eIF-5A pathway can be parasitized by HIV-1. HIV-1 now is being recognized as a non-limiting representative example of the class of viruses that, after penetration into eukaryotic cells, feed on the eIF-5A pathway to achieve preferential translation of viral structural proteins and thus, gain a generative advantage. This compatible with the finding that HIV-1 multiplication occurs preferentially in proliferating cells, particularly of the T-cell lineage >see, for instance, Gowda et aL, J. Immunol 142, 773-780 (1989) or Klatzmann et al., Immunol. Today 7, 291-296 (1986), and references therein, all of which are hereby incorporated by reference, and is compatible with the finding that efficient HIV replication in human peripheral blood mononucleolar cells and in human T-cell lines correlates with eIF-5A expression (Bevec et al., Proc. Natl. Acad. Sci. USA 91, 10829-10833 (1994), which is hereby incorporated by reference. A subclass of viral mRNAs encoding in particular the structural proteins that form the virion core and capsid, interacts with the viral protein Rev through a specific nucleotide sequence, the Rev response element (“RRE”). The Rev/RRE unit constitutes the specific regulator for biosynthesis of HIV-1 proteins (see, for instance, Gallo et al., The Human Retroviruses, 69-106, Academic Press (1991), which is hereby incorporated by reference. It is the Rev component of this complex which specifically interacts with host cell eIF-5A (Ruhl et al., J. Cell. BioI. 123, 1309-1320 (1993), which is hereby incorporated by reference. As a result, these RRE-containing viral mRNA species, which otherwise would show very limited or no translational efficiency, become eligible for preferential polysomal loading and translation, resulting in active production of infective HIV-1 virions. In this manner, the production of key proteins for virion formation and packaging is assured and viral replication guaranteed. Production of Rev at the host cell polysomes is known to occur independent of RRE, Rev and eIF-5A

The cellular partner for Rev/Rex is eukaryotic translation initiation factor 5A (eIF5A), of which two isoforms are known at present. Both contain the unique, genetically not encoded residue hypusine. Hypusine is essential for the biological function of the eIF5A proteins, i.e. the nucleocytoplasmic transport of proliferation-related cellular mRNAs. Hypusine is formed through two consecutive posttranslational modifications of a specific, genetically encoded lysine side chain. The last of these modifications is a hydroxylation, mediated by deoxyhypusine hydroxylase (DOHH a 2-oxoacid utilizing dioxygenase like all other known protein hydroxylases. In HIV infected cell lines, the 3-hydroxypyrid-4-one class of DOHH inhibitors deceases the formation of infective virions, disrupts the synthesis of the major capsid proteins, and induces apoptosis in the range of 100-200 μM.

Compositions of the present invention include a compound of formula (I) and derivatives thereof (salts, solvates, tautomeric forms),

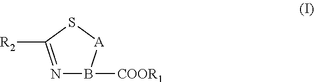

where

R₁ is hydrogen or a pharmacologically acceptable salt;

R₂ is ortho-hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen or hydroxyl; and

A-B is —CH₂—CR₃— or —CH═C—, where R₃ is hydrogen or (C₁-C₆)alkyl.

In one embodiment of the invention R₁ is hydrogen, R₂ is phenyl, A-B is —CH═CR₃— and R₃ is hydrogen. More preferably R₂ is 2-hydroxy-4-methylphenyl or 2,4-dihydroxyphenyl.

In another embodiment of the invention, R₁ is hydrogen and R₂ is pyryidyl. More preferably, R₂ is 3-hydroxy-2-pyridyl and A-B is —CH₂—C(CH₃)— or —CH═C—.

In another embodiment of the invention R₁ is hydrogen, R₂ is phenyl, A-B is —CH═C— and R₃ is hydrogen. More preferably R₂ is 2-hydroxy-4-methyl.

Examples of formula I useful in the practice of the present invention include but are not limited to rilopirox; ciclopirox, 6-cyclohexyl-l-hydroxy-4-methyl-2(1H)-pyridone; its ethanolamine salt ciclopirox olamine; metipirox which is 1-hydroxy-4,6-dimethyl-2-(1H)-pyridone (CAS #2934202-7); piroctone (1-hydroxy-4-methyl-6-(2,4,4-trimethylpentyl)-2-(1H)-pyridone [CAS #506050-76-5]); and its (1:1) ethanol amine salt octopirox (CAS #68890-6-4).

In another embodiment of the invention, compositions of the present invention include a compound of formula (II) and derivatives thereof (salts, solvates, tautomeric forms)

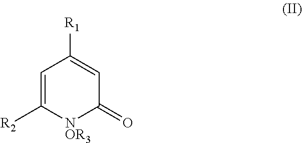

wherein

R₁ is (C₁-C₆) alkyl;

R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and

R₃ is hydrogen or a pharmacologically acceptable salt.

Preferably R₁ is methyl.

More preferably, R₁ is methyl, R₂ is cyclohexyl or

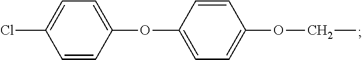

and

R₃ is hydrogen.

In another embodiment, R₁ is methyl, R₂ is (CH₃)₃CH₂C(CH₃)CH₂— and R₃ is ⁺H₃NCH₂OH.

In yet another embodiment of the invention, compositions of the present invention include a compound of formula (III) or (IV) and derivatives thereof (salts, solvates, tautomeric forms)

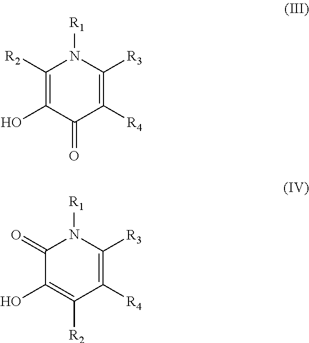

wherein R₁, R₂, R₃, and R₄ each individually represent a hydrogen, an alkyl, alkenyl or alkoxy group containing 1 to about 8 carbons, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

These compounds may be made by the methods disclosed in U.S. Pat. No. 2,540,218 and U.S. Pat. No. 4,797,409 the contents of which are incorporated herein by reference in their entirety.

The hydroxypyridones of the present invention may be used in the free form or as their physiologically tolerated salts with inorganic or organic bases such as but not limited to NaOH, KOH, Ca(OH)₂, NH₃, and H₂NCH₂CH₂OH.

For purposes of all of the present inventive methods, the amount or dose of the compound administered should be sufficient to effect a therapeutic response in the animal over a reasonable time frame. Particularly, the dose of any composition including one or more of the compounds of any of Formula [1-4] should be sufficient to inhibit formation hypusine in precursor protein of eIF-5A within about 24 hours. The dose will be determined by the efficacy of the particular compound and the condition of the patient, tissue, or cell sample as well as the mass of the sample or patient to be treated. Many assays for determining an administered dose are known in the art. For purposes of the present invention, an assay, which comprises comparing the extent to which viral proliferation is inhibited in a tissue or sample of cells upon administration of a given dose of a compound to set of samples that are each given a different dose of the compound, could be used to determine a starting dose to be administered. The extent to which cell apoptosis is restored upon administration of a certain dose can be assayed as known to those skilled in the art and described herein.

The size of the dose also will be determined by the existence, nature and extent of any adverse side effects that might accompany the administration of a particular compound. Ultimately, the attending physician will decide the dosage of the compound of the present invention with which to treat each individual patient, taking into consideration a variety of factors, such as age, body weight, general health, diet, sex, inhibitor to be administered, route of administration, and the severity of the condition being treated.

One skilled in the art will appreciate that suitable methods of administering a compound of the present invention are known, and, although more than one route can be used to administer a particular composition, a particular route can provide a more immediate and more effective response than another route.

Formulations suitable for oral administration of compositions which include compound of the present invention can consist of (a) liquid solutions, such as an effective amount of the compounds dissolved in diluents, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch potato starch, microcrystalline cellulose, acacia, gelatin, colloidal silicon dioxide, croscarmellose, sodium, talc, magnesium stearate, stearic acid, and other excipients, colorants, diluents, buffering agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

Formulations of the compounds of formula (1-4) in compositions suitable for parenteral administration include aqueous and non-aqueous solutions, isotonic sterile injection solutions, which can contain anti-oxidants, buffers,' bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

With respect to all of the present inventive methods, the 1-hydroxy-2-pyridones such as ciclopirox can be administered topically, systemically (intravenously or subcutaneously).

Furthermore, all of the present inventive methods can comprise the administration of the compound, in the presence or absence of an agent that enhances its efficacy, or the methods can further comprise the administration of other suitable components, such as radiation therapy or chemotherapy with another active agent. The term “radiation therapy” as used herein refers to the treatment of disease (especially cancer) by exposure to radiation. The term “chemotherapy” as used herein refers to the treatment of cancer using specific chemical agents or drugs that are destructive of malignant cells and tissues. Chemotherapy refers to the treatment of disease using chemical agents or drugs that are toxic to the causative agent of the disease, such as a virus, bacterium, or other microorganism.

Compounds of the present invention may be linked to radiological moieties such as ¹²⁵I for treatment of specific cancers.

If combined with radiation therapy or chemotherapy, the compounds of the present invention can be administered simultaneously or sequentially. The term “sequentially” as used herein refers to the compound being administered either before or after the radiation therapy or chemotherapy. Preferably, the compound is administered first, particularly if combined with radiation therapy.

One of ordinary skill in the art will readily appreciate that each compound of the present inventive methods can be modified in any number of ways, such that the therapeutic efficacy of the compound is increased through the modification. For instance, the compound could be conjugated either directly or indirectly through a linker to a targeting moiety. The practice of conjugating compounds to targeting moieties is known in the art. See, for instance, Wadwa et al., J. Drug Targeting 3: 111 (1995), U.S. Pat. No. 5,087,616, and U.S. Pat. No. 5,849,587 the contents of which are included herein by reference in their entirety. The term “targeting moiety” as used herein, refers to any molecule or agent that specifically recognizes and binds to a cell-surface receptor, such that the targeting moiety directs the delivery of the compound to a population of cells on which surface the receptor is expressed. Targeting moieties include, but are not limited to, antibodies, or fragments thereof, peptides, hormones, growth factors, cytokines, and any other naturally-or non-naturally-existing ligands, which bind to cell surface receptors. The term “linker” as used herein, refers to any agent or molecule that bridges the compound to the targeting moiety.

One of ordinary skill in the art recognizes that sites on the compounds, which are not necessary for the function of the compound, are ideal sites for attaching a linker and/or a targeting moiety, provided that the linker and or targeting moiety, once attached to the compound does not interfere with the function of the compound, i.e. the ability to inhibit formation of the hypusine residue and form eIF-5A.

Alternatively, the compounds of the present invention can be modified into a depot form, such that the manner in which the compound is released into the body to which it is administered is controlled with respect to time and location within the body (see, for example, U.S. Pat. No. 4,450,150). Depot forms of compounds can be, for example, an implantable composition comprising the compound and a porous material, such as a polymer, wherein the compound is encapsulated by or diffused throughout the porous material. The depot is then implanted into the desired location within the body and the compound is released from the implant at a predetermined rate by diffusing through the porous material.

The compounds of the present invention can be used to treat a number of viral diseases caused by viruses that require a specific mediator protein (i.e. Rev) or a functional equivalent) to express viral structural genes and to propagate efficiently. Such viruses include, but are not limited to, the lentiviruses pathogenic for humans and animals, in particular the human, bovine, feline, and simian immunodeficiency viruses, the equine infectious anemia virus, the caprine arthritis-encephalitis virus, and the visna virus.

In the practice of the present invention, compositions that include compounds a of formula (1) can be administered topically or systemically. More particularly, such administration can be orally; parenterally, i.e. by subcutaneous, intravascular, or intramuscular injection; intraperitoneally; intrathecally; or by topical application, e.g. to skin or eyes, or by application to the mucous membranes of the nose, throat, bronchial tree, or rectum, etc. They may be administered alone or with suitable pharmaceutical carriers, and can be in solid or liquid form such as tablets, capsules, powders, solutions, suspensions, or emulsions. The dosage of the active compound depends on the species of warm-blooded animal, the body weight age, and mode of administration.

The pharmaceutical products of the present invention are prepared by dissolving, mixing, granulating, or tablet-coating processes known to those skilled in the art. For oral administration, the active compounds or their physiologically tolerated derivatives such as salts, esters, or amides, are mixed with the additives customary for this purpose, such as vehicles, stabilizers, or inert diluents, and are converted by customary methods into a suitable form for administration, such as tablets, coated tablets, hard or soft gelatin capsules, aqueous, alcoholic, or oily suspensions, or aqueous, alcoholic or oily solutions. Examples of suitable inert vehicles are conventional tablet bases such as lactose, sucrose, or cornstarch in combination with binders like acacia, cornstarch, gelatin, or with disintegrating agents such as cornstarch, potato starch, alginic acid, or with a lubricant like stearic acid or magnesium stearate. Examples of suitable oily vehicles or solvents are vegetable or animal oils, such as sunflower oil or fish-liver oil. Preparations can be effected both as dry and as wet granules.

For parenteral administration (subcutaneous, intravascular, or intramuscular injection), the active compounds or their physiologically tolerated derivatives such as salts, esters, or amides, are converted into a solution, suspension, or emulsion, if desired, with the substances customary and suitable for this purpose, such as solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as propylene glycol or polyethylene glycol, are preferred liquid carriers, particularly for injectable solutions.

For use as aerosols, the active compounds or their physiologically tolerated derivatives such as salts, esters, or amides, may be dissolved or suspended in a physiologically acceptable liquid and packaged in a pressurized aerosol container together with suitable propellants, for example, hydrocarbon propellants like propane, butane, or isobutane with conventional adjuvants. The agents which block intracellular hypusine formation, in accordance with the present invention, may also be administered from a non-pressurized container such as a nebulizer or atomizer.

For topical administration to external or internal body surfaces, e.g., in the form of creams, gels, or drops, etc., the active compounds or their physiologically tolerated derivatives such as, salts, esters, or amides, are prepared and applied as solutions, suspensions, or emulsions in a physiologically acceptable diluent with or without a pharmaceutical carrier.

Various aspects of the present invention will be illustrated with reference to the following non-limiting examples.

### EXAMPLE 1

This example demonstrates that the 1-hydroxy-2-pyridone ciclopirox, formula (2)

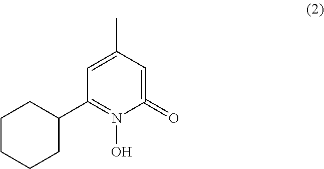

is about ten times more potent in causing a very similar spectrum of effects. Ciclopirox, is representative for the entire class of 1-hydroxy-2-pyridones, for instance with regard to their ability to form high-spin 1:3 complexes with d2sp3-hybridized metals. Test results repeatedly demonstrate that ciclopirox, causes a dose-dependent initiation of apoptosis in the HIV-1 infected lymphocyte cell line H9.

Exposure of these HIV-1 infected lymphocyte cell line H9 cells for a mere 18 hours (overnight) at 40 μM of (2) causes apoptosis in more than 30% of all cells. This effect is accompanied by a dose-dependent inhibition of cellular DOHH. By contrast, the agent does not trigger apoptosis in HPV infected human keratinocytes or in native human lymphocytes not infected by HIV-1. In these latter two cases, the virally infected and the normal cells are not kept alive by anti-apoptotic proteins whose nucelocytoplasmic transport and polysomal translation depend on a Rev and therefore eIF-5A/DOHH dependent mechanism.

The results show that selective ablation of HIV-1 infected cells may be achieved by disrupting the translation of retroviral anti-apoptotic proteins that are encoded by Rev-dependent mRNAs shuttled to those cell polysomes via the eIF-5A pathway of nuclear export.

Systemic application of the 1-hydroxy-2-pyridones may be used to treat cells, tissues, or patients having tissues with such viruses. The compositions may include the compound of formula (1) and their derivatives. The application may include cycled high dose pulse therapy administration of the composition to the cells, tissue, or patient to achieve ablation of lentiviruses, retroviruses, and preferably HIV infected cells throughout the body. Repeated treatment cycles with 1-hydroxy-2-pyridonense and their derivatives is possible, as are combinations with currently available chelators like desferal, to maximize the amount of a 1 hydroxy-2-pyridone like ciclopirox reaching DOHH, and to minimize the amount lost due to non-specific binding to metal ions, and or the currently available antiretrovirals, since none of the latter acts as an inhibitor of DOHH/the eIF-5A pathway.

### EXAMPLE 2

This prophetic example illustrates use of the compound of the present invention in compositions.

The compounds of the present invention may be included in compositions for topical applications. The 1-hydroxy-2-pyridones may be admixed into spermicidal creams, or coated into the lubricant of condoms, or used in preparations intended for pre/post-coital application. In such compositions the 1-hydroxy-2-pyridones may limit the production of infectious virons by lymphocytes in the ejaculate. Such lymphocytes appear to play a decisive role in genital transmission of HIV-1 which is premised on the fact that lymphocyte removal from the sperm of HIV-1 positive males during processing for in vitro fertilization results in non-infectious semen, as evidenced clinically by absent infection if inseminated females as well as by the generation of entirely healthy babies.

### EXAMPLE 3

This example illustrates the selective ablation of HIV-infected lymphocytes by inhibitors of hypusine formation.

Mature eIF5A, involved in nucleocytoplasmic transport of certain mRNAs, contains the functionally essential residue hypusine. The latter is fanned by deoxyhypusine hydroxylase (DOHH), a 2-oxoacid utilizing non-heme iron dioxygenase whose catalysis follows the HAG mechanism (Hanauske-Abel et al., Curr Med Chem 10: 1037-1050 (2003)). Mature eIF5A is a cellular cofactor of the viral Rev/Rex proteins (Hauber, Curr Top Microbiol Immunol 259:55-76 (2001 n, required for retroviral multiplication and suppression of host cell, but not metal chelators in general, deny mature eIF5A to HIV-1, causing a lack of retroviral anti-apoptotic proteins and thus releasing the self-destruction of FIN-infected lymphocytes. DOHH inhibition in human papillomavirus (HPV)-infected or normal cells should not trigger apoptosis.

Methods: The drug deferiprone (DEF) and ciclopirox (CPX), known as DOHH inhibitors, were compared to the chelators 2-imidazoyl-4-methylphenol (IMP) and desferal (DES). Apoptosis of HIV-1 infected H9 cells, HPV-16 infected SiHa cells, and uninfected lymphocytes was analyzed by TUNEL flow cytometry. DOHH) activity was measured by metabolic labeling with 3H-spennidine.

Results: In a dose-dependent manner, DEF and CPX inhibited DOHH in HIV-positive H9 cells and in HPV-positive SiHa cells. The chelator IMP was uniformly ineffective even at 400 11M. In the H9 cells, only the DOHH inhibitors DEF and CPX triggered dose-dependent apoptosis. The chelators IMP and DES failed to elicit apoptosis even at maximal concentrations (400 μM and 20 μM respectively). With complete suppression of DOHH activity, i.e. at 200 μM DEF or 40 μM CPX, at least 30% of these H9 cells became apoptotic within 20 hours. By contrast, 40 μM CPX did not initiate apoptosis in the SiHa cells even after 120 hours, although CPX totally suppressed their DOHH activity. Likewise, lymphocytes harvested from health volunteers failed to respond with apoptosis when exposed for 20 hours to DEF, CPX, or DES.

The results demonstrate the ability to chelate iron in solution is not sufficient for DOHH inhibition. The latter triggers apoptosis in HN-infection, but not in HPV-infected or normal cells. Deferiprone is a clinical trail candidate for a novel treatment strategy: Cycled, high-dose pulse therapy to achieve selective ablation of infected cells, bypassing all currently pursued and mutation-proneviral targets.

### EXAMPLE 4

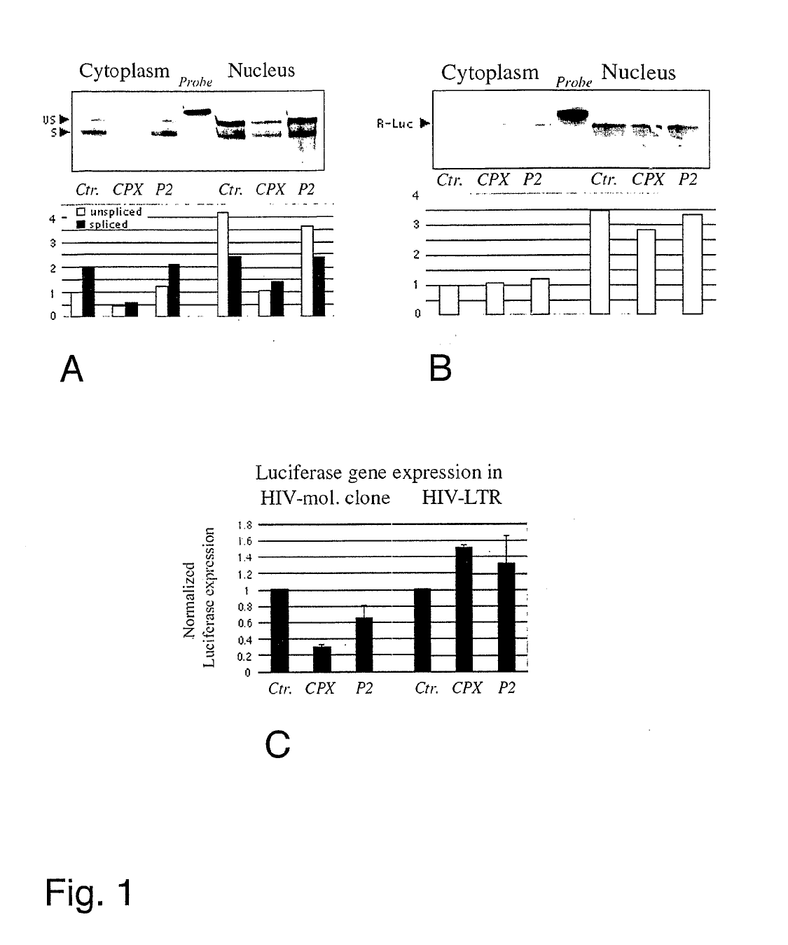

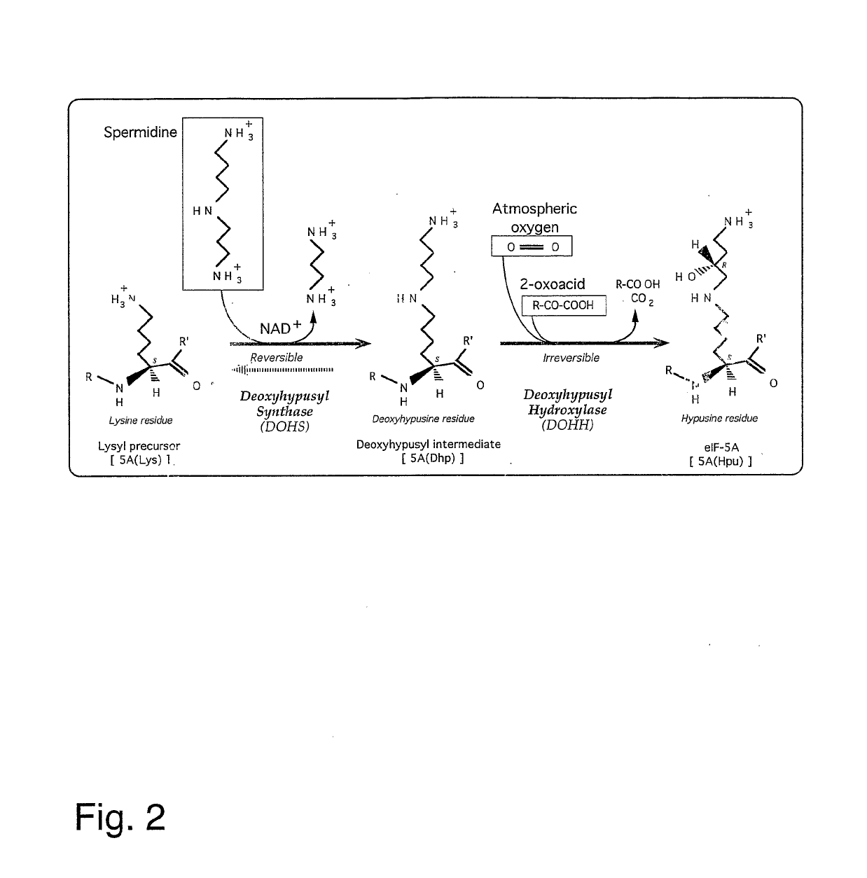

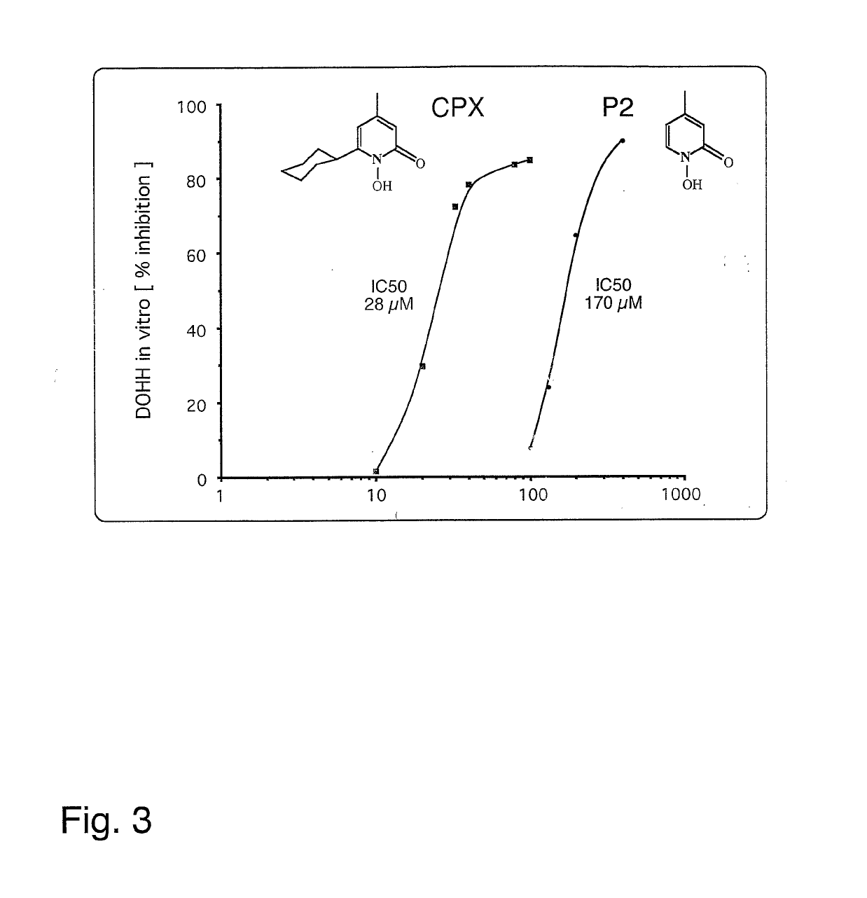

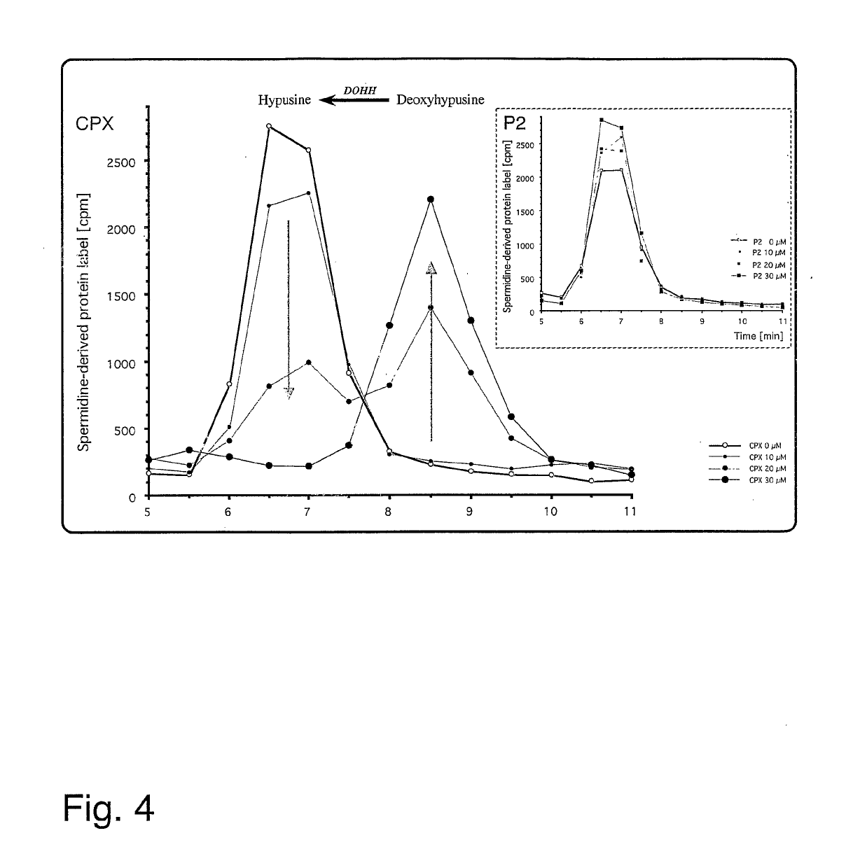

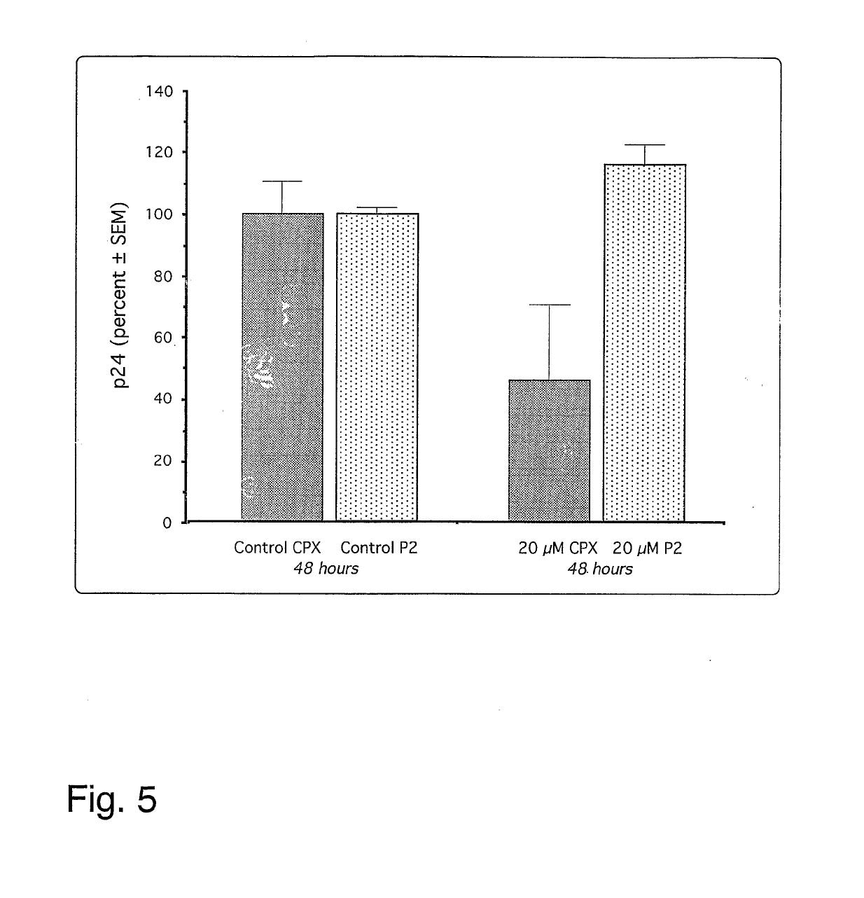

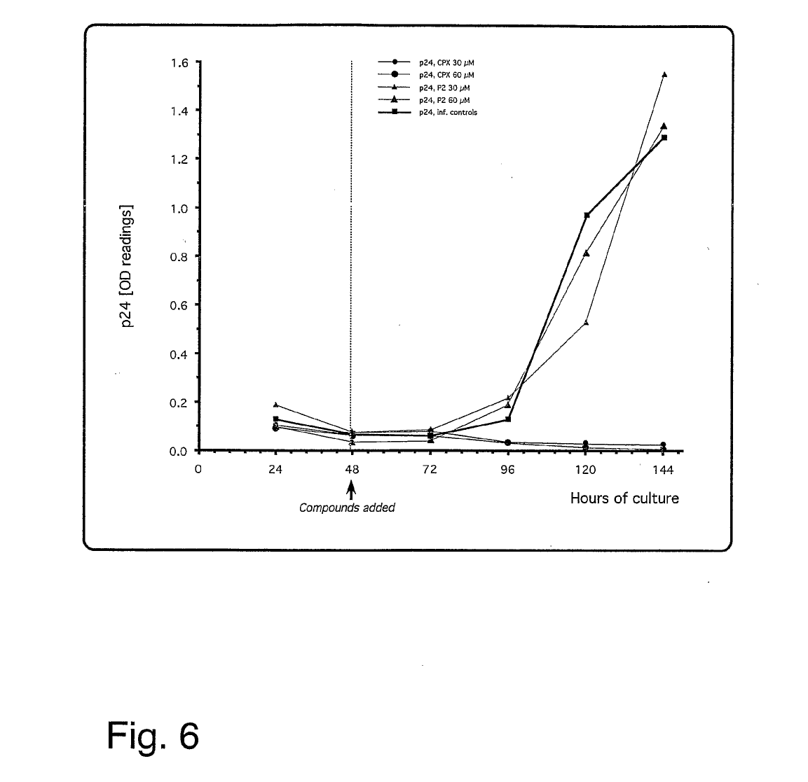

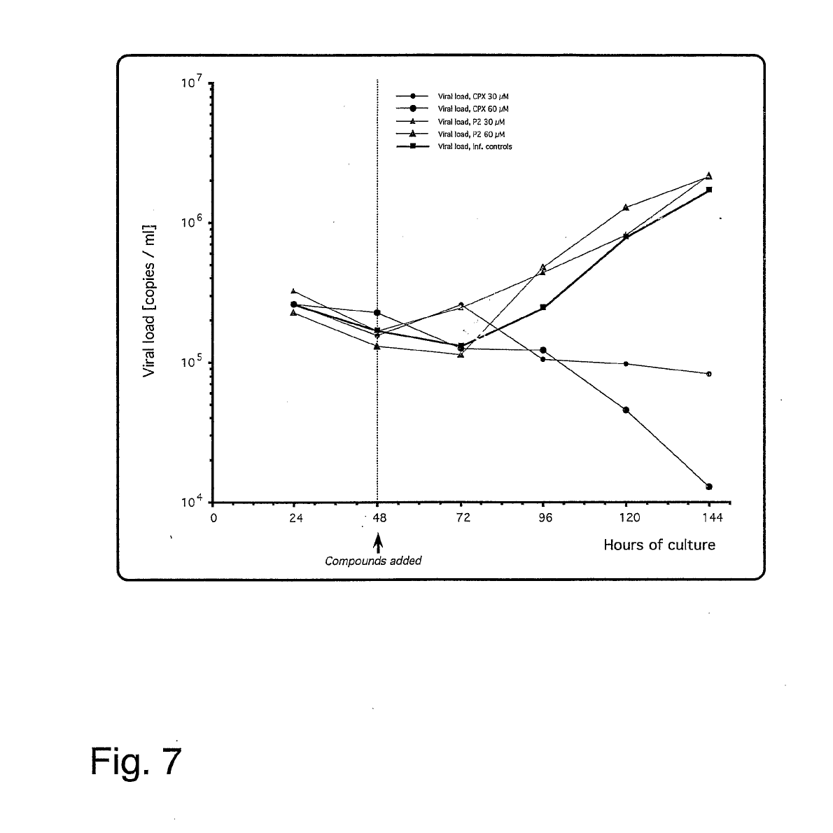

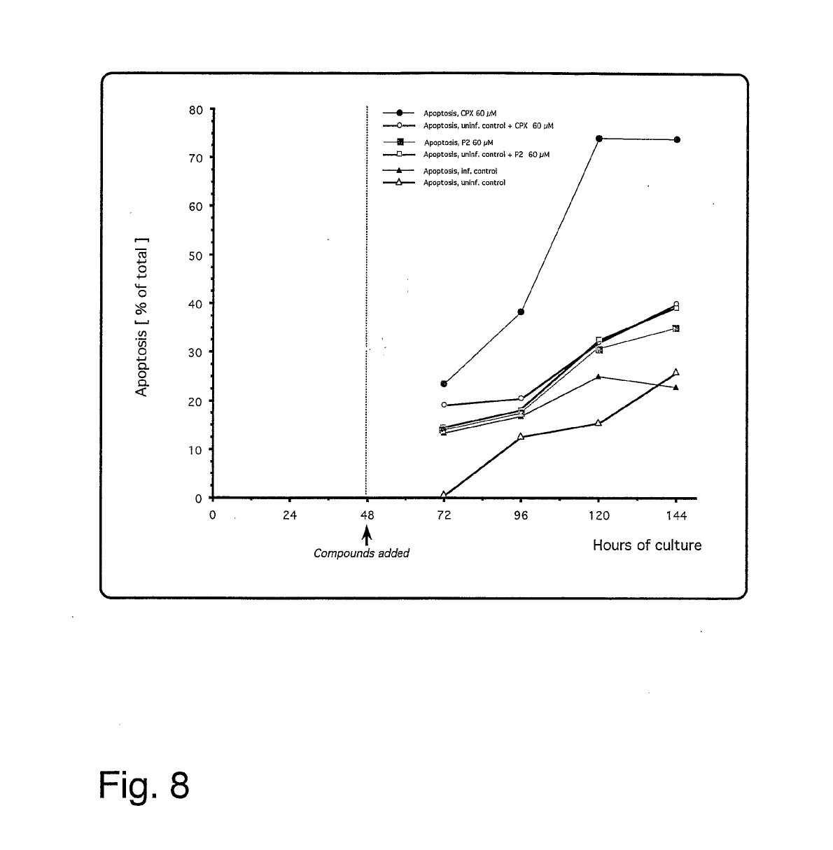

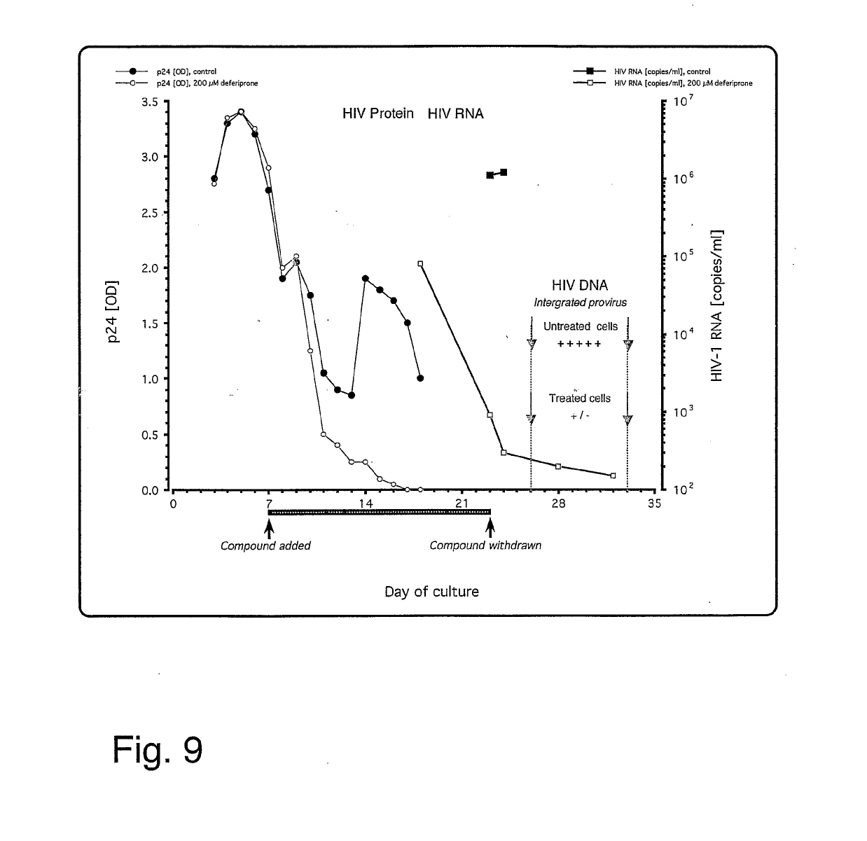

This example demonstrates that the N-hydroxy-2-pyridone ciclopirox, but not its analog P2, selectively suppresses the accumulation of sliced and unsliced transcripts encoded by human immunodeficiency virus 1 (HIV-1; FIG. 1A), whereas it does not affect transcript accumulation encoded by cytomegalovirus (CMV; FIG. 1B). Likewise, ciclopirox exerts a distinctly more inhibitory effect than P2 on the expression of a reporter gene, firefly luciferase, when encoded in a full-length, genomic HIV-1 clone (FIG. 1C, left side). Remarkably, and consistent with the requirement for the Rev-RRE system as partner of mature eIF5A to produce susceptibility to translational inhibition of hypusine formation, this suppressive effect was abrogated if the same reproter gene was not placed into the full-length, genomic HIV-1 clone, and instead placed next to merely the long terminal repeat (LTR) element of HIV-1. In this setting, which lacks the genetic RRE element and thus dependency on hypusine formation, ciclopirox did not reduce luciferase gene expression compared to control and P2 (FIG. 1C, right side). The suppression of retroviral protein synthesis by ciclopirox (FIG. 1C, left side), and thus by retroviral proteins optimizing retroviral transcription, may in turn result in inefficient synthesis of retroviral RNA (FIG. 1A).

For this set of experiments, the following procedures and reagents were used:

Cells and plasmids. 293T cells were grown in Dulbecco's Modified Eagle's medium with 10% fetal bovine serum. pBSIIKS+HIV(+80-341) was constructed by subcloning the PCR-amplified HIV-1 sequence corresponding to nucleotides +80 to +341 of the HIV molecular clone (pNL4-3-Luc E-) between the SmaI and HindIII sites of the pBluescriptllKS+ vector (Stratagene, Calif.). Plasmids pSP-rluc and pCMV-Renilla were purchased from Promega (Madison, Wis.). The plasmids pRSV-Tat and pLTR-firefly luciferase were described earlier (Hogue et al. MCB 2003). The HIV-1 molecular clone pNL4-3-Luc E-, in which part of the Nef gene is replaced by firefly luciferase and the envelope gene is mutated (Chen B. K., J. Virol. 1994), was generously supplied by Dr. D. Baltimore.

Gene expression assays. RNase protection assays were performed with 10 μg of cytoplasmic RNA and 5 μg of nuclear RNA, isolated from transfected 293T cells as described previously (Young et al 2003 MCB), using the RPAIII kit (Ambion, Austin, Tex.) according to the manufacturer's instructions. Probes were generated from pBSII-KS+HIV(+80-341) and pSP-rluc linearized with HindIII and BsaA1, respectively, by transcription with T7 RNA polymerase in the presence of [alpha-³²P]UTP (ICN Pharmaceuticals Inc., Costa Mesa, Calif.). The resulting HIV splice junction and Renilla probes were 330 and 245 nucleotides, respectively. The protected fragments for unspliced and spliced HIV RNA are 261 and 208 nucleotides, respectively, and that for Renilla luciferase 186 nucleotides. Firefly and Renilla luciferase assays were performed with the dual luciferase reporter system (Promega) according to the manufacturer's instructions.

Consistent with these findings is the fact that purified DOHH, the hypusine-forming enzyme, is disctinctly more susceptible to inhibition by ciclopirox (CPX) than by its analog Princeton 2 (P2), as shown in FIG. 3. The assay was conducted by using purified enzyme and offering deoxhypusine-containing, deoxhypusine-labeled eIF5A as substrae, plus the appropriate cofactors (see FIG. 2). Enzyme activity was assessed by determining the amount of mature, ie. radioactive hypusine-containing eIF5A via protein hydrolysis, followed by amino acid analysis.

### EXAMPLE 5

As shown in FIG. 4, exposure of the HIV-1-infected T-lymphocyte cell line H9 to ciclopirox causes a dose-dependent inhibition of their DOHH activity. By contrast, P2 is entirely non-inhibitory even at the highest concentration tested (FIG. 4, inset). DOHH activity was measured by metabolic labeling with radioactive spermidine (see FIG. 2), followed by protein hydrolysis\ and amino acid analysis as reported (Hanauske-Abel et al., FEBS Left. 366, 92-98, 1995, which is hereby incorporated by reference). This finding corroborates the results obtained with purified DOHH (FIG. 3) and is consistent with the findings presented in FIG. 1.

In the same HIV-1 infected cells, ciclopirox but not its analog P2, suppressed retroviral protein synthesis, as assessed via p24 (FIG. 5). The latter was measured by commercial immunoassay. This finding is consistent with incapacitation of mature eIF5A, the cellular partner for the Rev-RRE system of HIV-1, and concordant with the results obtained with purified (FIG. 3) and cellular (FIG. 4) DOHH. The ciclopirox-mediated suppression of the retrovirally encoded p24 antigen (FIG. 5) and concurs with the ciclopirox-mediated suppression of the retrovirally encoded luciferase reproter gene (FIG. 1C, left side).

### EXAMPLE 6

As shown in FIG. 6, ciclopirox but not its analog P2 is efficient in suppressing the formation of retroviral p24 protein in freshly isolated peripheral blood mononuclear cells, acutely infected with a patient-isolate of HIV-1. This system is considered a close model for the actual infection of a human being by ‘wild-type’ HIV-1.

As shown in FIG. 7, ciclopirox but not its analog P2 is efficient in suppressing the formation of retroviral RNA, determined as viral load [copies/ml] by polymerase chain reaction with specific primers, in freshly isolated peripheral blood mononuclear cells, acutely infected with a patient-isolate of HIV-1. This system is considered a close model for the actual infection of a human being by ‘wild-type’ HIV-1.

As shown in FIG. 8, ciclopirox but not its analog P2 triggers apoptosis in freshly isolated peripheral blood mononuclear cells preferentially when acutely infected with a patient-isolate of HIV-1. This effect is ascribed to the suppression of retroviral antiapoptotic proteins as part of the general suppression of retroviral protein synthesis, as exemplified by the decrease in p24 (FIG. 6).

### EXAMPLE 7

The double y-axis plot of FIG. 9 summarizes the effect of deferiprone on HIV protein synthesis (p24 [left y-axis]), HIV RNA synthesis (copies [right y-axis]), and the presence of HIV DNA, integrated as provirus into the genome of the infected host cells. The cell system and mode of infection used are the same as in FIGS. 6 to 8, i.e. freshly isolated peripheral blood mononuclear cells infected with a patient-isolate of HIV-1, except that the compound was added only after the full-blown infection had developed. The design requires the addition of uninfected cells very second day, so as to keep the infection from burning out. At the time when p24 became undetectable, HIV RNA was still measurable, but dropped to the limit of detection over the next days and did not increase even after discontinuation of the compound. At the end of the representative experiment, the deferiprone-treated cultured cells have at best a trace of integrated provirus and HIV RNA, whereas the untreated cells display high levels of both integrated provirus and HIV RNA. The near-eradication of provirus is attributed to the selective apoptotic ablation of HIV-infected cells by DOHH inhibitors (see FIG. 8).

Although the present invention has been described in considerable detail with reference to certain preferred embodiments thereof, other versions are possible. For example While not wishing to be bound by theory, since the compounds of the general formula (1), according to this invention, inhibit the enzymatically catalyzed hydroxylations of proteins, they are apt to prevent the maturation of such proteins which do not become biologically functional until in their hydroxylated forms. These hydroxylation-dependent proteins are, for instance, the collagens, the ribosomal initiation factor eIF-5A, and LTBP, the chaperone for synthesis of bioactive TGF-B. If their hydroxylation is suppressed by inhibition of the enzymes which catalyze this reaction, i.e. prolyl 4-hydroxylase, deoxyhypusyl hydroxylase, and aspartyl/asparaginyl hydroxylase, respectively, these proteins are rendered unable to function. As the functions of these hydroxylation-dependent proteins converge in the clinical disease group of fibrotic and fibroproliferative conditions, the protein hydroxylase inhibitors of Formula (I-IV) may be suitable instruments to control and treat such conditions pharmacologically. Therefore the spirit and scope of the appended claims should not be limited to the description and the preferred versions contain within this specification.

1. A method of inhibiting intracellular translation of viral mRNAs into viral proteins required for virion assembly and infectivity, comprising:
administering, to eukaryotic cells, tissues, or individuals, an agent which blocks the accumulation of spliced and unspliced viral transcripts and their utilization for viral protein synthesis at cellular ribosomes.

2. The method of claim 1 wherein the agent is administered topically.

3. The method of claim 1 wherein the agent comprises a compound of formula (I) 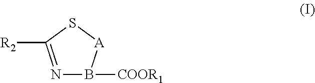
where
R₁ is hydrogen or a pharmacologically acceptable salt;
R₂ is ortho-hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen or hydroxyl; and
A-B is —CH₂—CR₃— or —CH═C—, where R₃ is hydrogen or (C₁-C₆)alkyl.

4. The method of claim 3 wherein R₁ is hydrogen, R₂ is phenyl, A-B is —CH═CR₃— and R₃ is hydrogen.

5. The method of claim 3 wherein R₁ is hydrogen and R₂ is pyridyl.

6. The method of claim 3 wherein R₁ is hydrogen, R₂ is phenyl, A-B is CH═C— and R₃ is hydrogen.

7. The method of claim 1 wherein the agent comprises a compound of formula (II)

8. 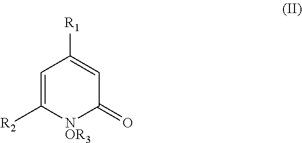
wherein
R₁ is (C₁-C₆) alkyl;
R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and
R₃ is hydrogen or a pharmacologically acceptable salt.

8. The method of claim 7 wherein R₁ is methyl.

9. A method of inhibiting the utilization of spliced and unspliced viral transcripts for viral protein synthesis at cellular ribosomes comprising:
administering, to eukaryotic cells, tissues, or individuals, an agent which blocks hypusine formation within eIF5A in an amount sufficient to suppress the translationally productive interaction of eIF-5A with viral elements of nucleic acid and/or protein structure.

10. The method of claim 9 wherein the agent is administered topically.

11. The method of claim 9 wherein the agent comprises a compound of formula (I) 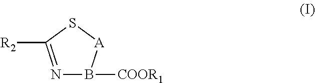
where
R₁ is hydrogen or a pharmacologically acceptable salt;
R₂ is ortho-hydroxy-substituted phenyl or pyridyl, where the phenyl or pyridyl group is otherwise unsubstituted or substituted with 1 to 3 additional substituents selected from the group consisting of (C₁-C6) alkyl, phenyl, (C₁-C₆)alkoxy, halogen or hydroxyl; and
A-B is —CH₂—CR₃— or —CH═C—, where R₃ is hydrogen or (C₁-C₆)alkyl.

12. The method of claim 11 wherein R₁ is hydrogen, R₂ is phenyl, A-B is —CH═CR₃— and R₃ is hydrogen.

13. The method of claim 11 wherein R₁ is hydrogen and R₂ is pyridyl.

14. The method of claim 11 wherein R₁ is hydrogen, R₂ is phenyl, A-B is CH═C— and R₃ is hydrogen.

15. The method of claim 9 wherein the agent comprises a compound of formula (II) 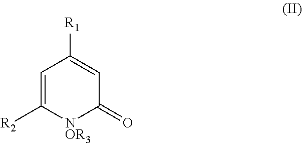
wherein
R₁ is (C₁-C₆) alkyl;
R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and
R₃ is hydrogen or a pharmacologically acceptable salt.

16. The method of claim 15 wherein R₁ is methyl.

17. A method of inhibiting synthesis of specific viral proteins of Rev/Rex-dependent lentiviruses, or of viruses dependent on interaction of eIF-5A with viral elements of nucleic acid and/or protein structure comprising:
administering, to eukaryotic cells, tissues, or individuals, an agent which blocks hypusine formation and thus eIF5A function in an amount sufficient to inhibit biosynthesis of viral proteins of Rev/Rex-dependent lentiviruses or of viruses dependent on interaction of eIF-5A with viral elements of nucleic acid and/or protein structure.

18. The method of claim 17 wherein the agent is administered topically.

19. The method of claim 17 wherein the agent comprises a compound of formula (I) 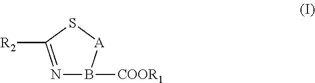
where
R₁ is hydrogen or a pharmacologically acceptable salt;
R₂ is ortho-hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen or hydroxyl; and
A-B is —CH₂—CR₃— or —CH═C—, where R₃ is hydrogen or (C₁-C₆)alkyl.

20. The method of claim 17 wherein R₁ is hydrogen, R₂ is phenyl, A-B is —CH═CR₃— and R₃ is hydrogen.

21. The method of claim 17 wherein R₁ is hydrogen and R₂ is pyridyl.

22. The method of claim 17 wherein R₁ is hydrogen, R₂ is phenyl, A-B is CH═C— and R₃ is hydrogen.

23. The method of claim 15 wherein the agent comprises a compound of formula (II) 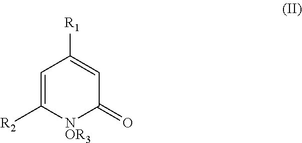
wherein
R₁ is (C₁-C₆) alkyl;
R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and
R₃ is hydrogen or a pharmacologically acceptable salt.

24. The method of claim 23 wherein R₁ is methyl.

25. A method of inhibiting replication of Rev/Rex-dependent lentiviruses, or viruses dependent on interaction of eIF-5A with viral elements of nucleic acid and/or protein structure comprising:
administering, to eukaryotic cells, tissues, or individuals, an agent which blocks hypusine formation and thus eIF5A function or reduces the availability of Rev/Rex protein, in an amount sufficient to inhibit replication of Rev/Rex-dependent lentiviruses or of viruses dependent on interaction of eIF-5A with viral elements of nucleic acid and/or protein structure.

26. The method of claim 25 wherein the agent is administered topically.

27. The method of claim 25 wherein the agent comprises a compound of formula (I) 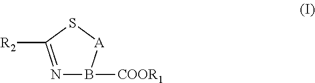
where
R₁ is hydrogen or a pharmacologically acceptable salt;
R₂ is ortho-hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen or hydroxyl; and
A-B is —CH₂—CR₃— or —CH═C—, where R₃ is hydrogen or (C₁-C6)alkyl.

28. The method of claim 27 wherein R₁ is hydrogen, R₂ is phenyl, A-B is —CH═CR₃— and R₃ is hydrogen.

29. The method of claim 27 wherein R₁ is hydrogen and R₂ is pyridyl.

30. The method of claim 27 wherein R₁ is hydrogen, R₂ is phenyl, A-B is CH═C— and R₃ is hydrogen.

31. The method of claim 25 wherein the agent comprises a compound of formula (II) 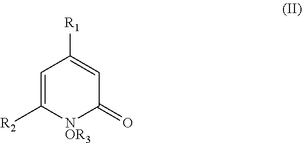
wherein
R₁ is (C₁-C₆) alkyl;
R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and
R₃ is hydrogen or a pharmacologically acceptable salt.

32. The method of claim 31 wherein R₁ is methyl.

33. A method of inducing apoptosis in cells infected with Rev/Rex-dependent lentiviruses or viruses dependent on interaction of eIF-5A with viral elements of nucleic acid and/or protein structure comprising:
administering, to cells infected with such viruses, an agent which blocks intracellular hypusine formation or reduces the availability of Rev/Rex protein, in an amount sufficient to induce apoptotic ablation of virally-infected cells.

34. The method of claim 33 wherein the agent is administered topically.

35. The method of claim 33 wherein the agent comprises a compound of formula (I) 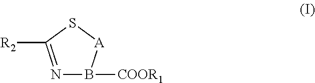
where
R₁ is hydrogen or a pharmacologically acceptable salt;
R₂ is ortho-hydroxy-substituted phenyl or pyridyl, where the phenyl or pyridyl group is otherwise unsubstituted or substituted with 1 to 3 additional substituents selected from the group consisting of (C₁-C₆) alkyl, phenyl, (C₁-C₆)alkoxy, halogen or hydroxyl; and
A-B is —CH₂—CR₃— or —CH═C—, where R₃ is hydrogen or (C₁-C₆)alkyl.

36. The method of claim 35 wherein R₁ is hydrogen, R₂ is phenyl, A-B is —CH═CR₃— and R₃ is hydrogen.

37. The method of claim 35 wherein R₁ is hydrogen and R₂ is pyridyl.

38. The method of claim 35 wherein R₁ is hydrogen, R₂ is phenyl, A-B is CH═C— and R₃ is hydrogen.

39. The method of claim 35 wherein the agent comprises a compound of formula (II) 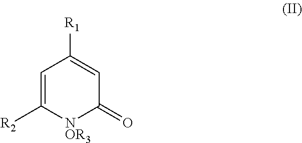
wherein
R₁ is (C₁-C₆) alkyl;
R₂ is (C₁-C₁₀) straight or branched alkyl, (C₃-C₆)cycloalkyl or phenoxy(C₁-C₃)alkyl, where the phenoxy group is substituted by substituted or unsubstituted phenoxy; and
R₃ is hydrogen or a pharmacologically acceptable salt.

40. The method of claim 31 wherein R₁ is methyl.

41. A method according to claim 1, wherein said administering is carried out topically or systemically.

42. A method according to claim 1 wherein said administering is carried out by percutaneous, oral, intravascular, intramuscular, intraperitoneal, intrathecal, or subcutaneous application, or ocular and mucous membrane administration.

43. A method according to claim 27, wherein the Rev-dependent lentivirus or virus dependent on interaction of host cell eIF-5A with viral elements of nucleic acid and/or protein structure, is selected from the group consisting of the human immunodeficiency viruses, the human T-cell leukemia viruses, the hepatitis B virus, the simian immunodeficiency viruses, the bovine immunodeficiency viruses, the feline immunodeficiency viruses, visna virus, equine infectious anemia virus, caprine arthritis-encephalitis virus, and Mason-Pfizer virus.

44. A method according to claim 43, wherein said method is used to inhibit human immunodeficiency viruses.

45. A method for suppressing genital transmission of human immunodeficiency virus which comprises administering to a male or female genital a compound of formula III or IV 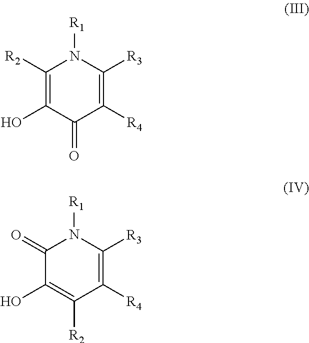
wherein R₁, R₂, R₃, and R₄ each individually represent a hydrogen, an alkyl, alkenyl or alkoxy group containing 1 to about 8 carbons, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

46. The method of claim 45 wherein the compound is deferiprone.
